(19) United States
(12) Patent Application Publication
(10) Pub. No.: US 2016/0135688 A1
(43) Pub. Date: May 19, 2016

(54) OBJECT INFORMATION ACQUIRING APPARATUS

(71) Applicant: CANON KABUSHIKI KAISHA, Tokyo (JP)

(72) Inventor: Hisafumi Ebisawa, Tokyo (JP)

(21) Appl. No.: 14/939,003

(22) Filed: Nov. 12, 2015

(30) Foreign Application Priority Data
Nov. 19, 2014 (JP) 2014-234754

(51) Int. Cl. A61B 5/00

(52) U.S. Cl. CPC A61B 5/0095; A61B 5/4312; A61B 5/7278; A61B 5/708

(57) ABSTRACT

Provided is an object information acquiring apparatus including: a holding member that has a curvature and holds an object; a conversion element that receives an acoustic wave propagating from the object via the holding member and outputs an electrical signal; a driving unit that moves at least one of the conversion element and the holding member so that an acoustic wave reception surface of the conversion element is not parallel to a tangential plane of the holding member at a position at which a normal line of the acoustic wave reception surface drawn from the conversion element crosses the holding member; and an information processor that acquires specific information inside the object using the electrical signal.

### BACKGROUND OF THE INVENTION

1. Field of the Invention

The present invention relates to an object information acquiring apparatus.

2. Description of the Related Art

Conventionally, an X-ray mammography apparatus for examining the breast to diagnose a breast cancer has been used widely. This apparatus presses and holds the breast which is an object using a holding member, irradiates the breast with X-rays, detects object information using a detector, and converts the object information into an image. However, the X-ray mammography apparatus has the problem of radiation exposure.

Thus, an apparatus which does not have the problem of radiation exposure is attracting attention. For example, an ultrasound imaging apparatus that irradiates an object with ultrasound waves and converts an echo signal from the object into an image is known. Moreover, a photoacoustic imaging apparatus that irradiates a living object with light from a light source such as a laser and converts photoacoustic waves from the object generated based on the incident light into images is also known. Japanese Patent Application Laid-open No. 2012-179348 discloses a photoacoustic imaging apparatus that scans a photoacoustic probe on an object held by a cup-shaped holding member to acquire photoacoustic waves and converts object information into a three-dimensional image from the acquired photoacoustic waves.

Patent Literature 1: Japanese Patent Application Laid-open No. 2012-179348

### SUMMARY OF THE INVENTION

FIG. 13A illustrates a schematic configuration of the apparatus disclosed in Japanese Patent Application Laid-open No. 2012-179348. In the drawing, an object 001 which is a measurement target is held by a cup-shaped holding member 002 having a curvature. When a light source 027 irradiates the object 001 with light, photoacoustic waves are generated, which propagate through a matching material 005. The photoacoustic waves are received by a plurality of conversion elements 004 supported by a hemispherical photoacoustic probe 026. In this case, by scanning the photoacoustic probe 026 on the object, a wide range of areas can be imaged.

The photoacoustic imaging apparatus is ideal for imaging a light absorber having a large optical absorption coefficient such as blood vessels inside an object. On the other hand, it is difficult to image the cancer itself as well as newborn blood vessels. Thus, the apparatus of FIG. 13B is provided with an ultrasound probe 003 which is disposed inside a support and can be scanned by a driving mechanism 008. Since this photoacoustic imaging apparatus also serves as an ultrasound imaging apparatus capable of detecting a structure such as a cancer, the apparatus can display an ultrasound image and a photoacoustic image in a superimposed manner to generate an image useful for diagnosis.

However, in the apparatus of FIG. 13B, depending on a scanning position of the ultrasound probe 003, a surface (element plane) in which the conversion elements of the probe are arranged and ultrasound waves are transmitted and received may be parallel to a tangential plane of the holding member positioned in the direction normal to the element plane. In this case, as illustrated in FIG. 13C, multiple reflections occur between the element plane and the holding member and appear on an image as artifacts.

In view of the above problems, it is an object of the present invention to provide an object information acquiring apparatus that allows a probe to scan an object held by a holding member and is capable of suppressing multiple reflections occurring between an element plane of the probe and the holding member.

The present invention provides an object information acquiring apparatus comprising:

a holding member configured to have a curvature and hold an object;

a conversion element configured to receive an acoustic wave propagating from the object via the holding member and output an electrical signal;

a driving unit configured to move at least one of the conversion element and the holding member so that an acoustic wave reception surface of the conversion element is not parallel to a tangential plane of the holding member at a position at which a normal line of the acoustic wave reception surface drawn from the conversion element crosses the holding member; and

an information processor configured to acquire specific information inside the object using the electrical signal.

According to the aspects of the present invention, it is possible to provide an object information acquiring apparatus that allows a probe to scan an object held by a holding member and is capable of suppressing multiple reflections occurring between an element plane of the probe and the holding member.

Further features of the present invention will become apparent from the following description of exemplary embodiments with reference to the attached drawings.

### BRIEF DESCRIPTION OF THE DRAWINGS

FIG. 1 is a diagram illustrating a configuration of an object information acquiring apparatus;

FIG. 2 is a diagram illustrating a configuration of a signal processor;

FIGS. 3A and 3B are diagrams illustrating a layout of a probe and conversion elements and a conversion element group plane;

FIG. 4 is a diagram illustrating an inclination angle between the probe and each imaging region during driving of the probe;

FIGS. 5A to 5E are diagrams how an acoustic wave propagates between a conversion element group and a holding member;

FIGS. 6A to 6E are diagrams illustrating an arrangement of acquired three-dimensional image data in relation to a layout of an object and the probe;

FIGS. 7A to 7D are schematic diagrams of an object information acquiring apparatus according to a first embodiment;

FIGS. 8A and 8B are diagrams illustrating a superimposed region of three-dimensional images acquired from different scanning regions;

FIG. 9 is a schematic diagram of an object information acquiring apparatus according to a second embodiment;

FIGS. 10A and 10B are schematic diagrams of an object information acquiring apparatus according to a third embodiment;

FIG. 11 is a schematic diagram of an object information acquiring apparatus according to a fourth embodiment;

FIG. 12 is a schematic diagram of an object information acquiring apparatus according to a fifth embodiment of the present invention; and

FIGS. 13A to 13C are schematic diagrams of the apparatus of the related art and are diagrams for describing the problem of the related art.

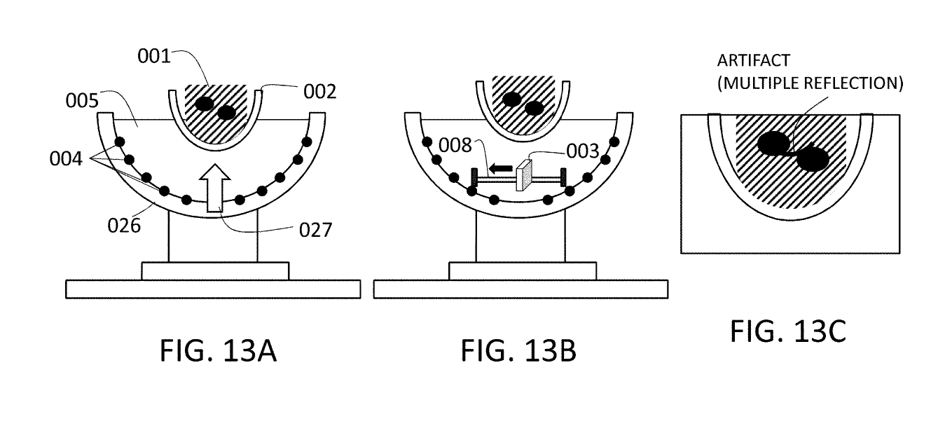

### DESCRIPTION OF THE EMBODIMENTS

Hereinafter, preferred embodiments of the present invention will be described with reference to the drawings. Dimensions, materials, shapes, relative arrangements, and the like of constituent components described below are to be appropriately changed according to the configuration and various conditions of an apparatus to which the present invention is applied, and the scope of the present invention is not limited to those described below.

The present invention relates to a technique of detecting acoustic waves having propagated from an object to generate and acquire specific information inside the object. Thus, the present invention can be understood as an object information acquiring apparatus or a control method thereof, and alternatively, as an object information acquiring method and a signal processing method. Moreover, the present invention can be understood as a program for allowing an information processing apparatus having hardware resources such as a CPU to execute these methods and a storage medium having the program stored therein. Further, the present invention can be understood as an acoustic wave measurement apparatus and a control method thereof.

The object information acquiring apparatus according to the present invention includes an apparatus which uses a photoacoustic tomography technique to irradiate an object with light (electromagnetic waves) to receive (detect) acoustic waves having propagated through the object after being generated inside or on the surface of the object according to a photoacoustic effect. Such an object information acquiring apparatus can be referred to as a photoacoustic tomography apparatus and a photoacoustic imaging apparatus in that specific information inside the object is obtained in a format such as image data based on photoacoustic measurement.

The specific information in the photoacoustic apparatus indicates a generation source distribution of the acoustic waves generated by light irradiation, an initial sound pressure distribution inside the object, an optical energy absorption density distribution and an absorption coefficient distribution derived from the initial sound pressure distribution, or a concentration distribution of a substance that constitutes a tissue. Specific examples of the specific information include a blood component distribution such as oxygenated and reduced hemoglobin concentration distributions or an oxygen saturation distribution derived from the distributions, and a distribution of fats, collagen, and water. Moreover, the specific information may be obtained as distribution information at respective positions inside an object rather than as numerical data. That is, distribution information such as an absorption coefficient distribution or an oxygen saturation distribution may be used as object information.

The acoustic wave acquiring apparatus of the present invention includes an apparatus which uses an ultrasound echo technique of transmitting ultrasound waves to an object and receives reflection waves (echo waves) reflected inside the object to acquire object information as image data. In the case of an apparatus which uses the ultrasound echo technique, the acquired object information is information that reflects a difference in acoustic impedance of tissues inside the object.

Acoustic waves referred in the present invention are typically ultrasound waves, and include elastic waves called sound waves and acoustic waves. The acoustic waves generated by the photoacoustic effect are referred to as photoacoustic waves or light-induced ultrasound waves. Electrical signals converted from acoustic waves by a probe are also referred to as acoustic signals, and acoustic signals originating from photoacoustic waves are referred to as photoacoustic signals in particular.

The breast of a living body can be considered as an example of the object used in the present invention. The object is not limited thereto, and other segments of a living body and non-living materials can be also measured.

(Apparatus Configuration)

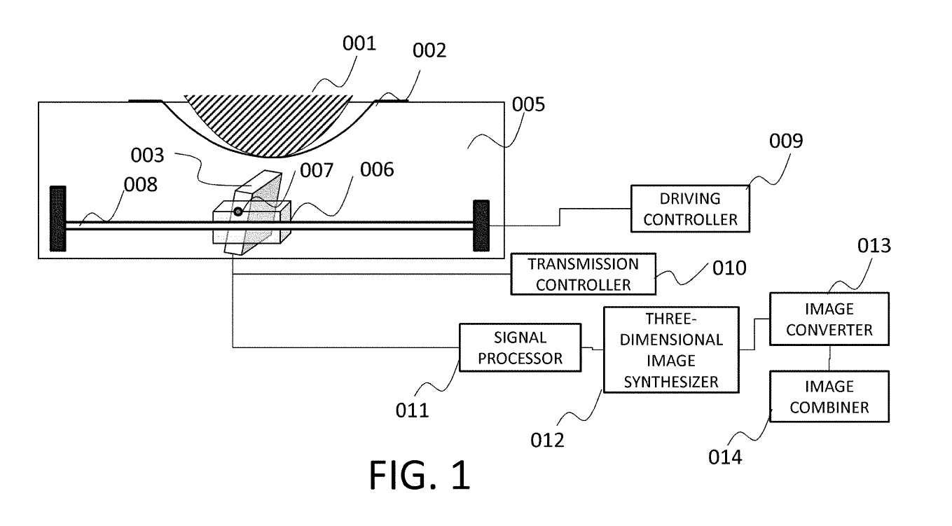

An example of an object information acquiring apparatus of the present invention will be described with reference to FIG. 1. This apparatus is an ultrasound imaging apparatus that holds an object using a hemispherical cup-shaped holding member, scans the probe vertically and horizontally on the holding member, and acquires object information of an imaging region. An information processing apparatus or circuit and a display apparatus that process signals and images may be considered to be included in the ultrasound imaging apparatus and may be considered to be provided separately from the ultrasound imaging apparatus and combined with the ultrasound imaging apparatus to form a system.

Reference numeral 001 is an object of which the image is acquired, and reference numeral 002 is a holding member that holds the object 001. Reference numeral 003 is an ultrasound probe having a function of transmitting ultrasound waves and a function of detecting ultrasound waves (echo waves) reflected from the object to output an electrical signal. The ultrasound probe 003 includes a number of conversion elements 004. A matching material 005 that propagates acoustic waves is present between the probe 003 and the holding member 002. The probe 003 is fixed to a carriage 006, and an inclination adjustment mechanism 007 for adjusting an attachment angle of the probe 003 is provided in the carriage 006. Reference numeral 008 is a driving mechanism that moves the carriage 006 on a two-dimensional plane. Reference numeral 009 is a driving controller that controls the driving mechanism 008. The driving control may not necessarily involve movement along the two-dimensional plane exactly but may involve movement along the z-direction.

Reference numeral 010 is a transmission controller that controls driving timings for aligning a transmission focus of ultrasound waves at an arbitrary position of the object 001. Reference numeral 011 is a signal processor that amplifies and AD-converts an electrical signal originating from the ultrasound echoes from the object and acquires (reconstructs images) specific information inside the object based on the electrical signal. Reference numeral 012 is a three-dimensional image synthesizer that creates a three-dimensional reconstruction image based on the coordinate of the probe 003 scanned by the driving mechanism 008. Reference numeral 013 is an image converter that corrects a three-dimensional image distorted by an inclination angle set by the inclination adjustment mechanism 007 to an actual space coordinate system. Reference numeral 014 is an image combiner that combines converted images corrected to the actual space coordinate system by the image converter 013 to create a three-dimensional ultrasound image of an entire imaging region.

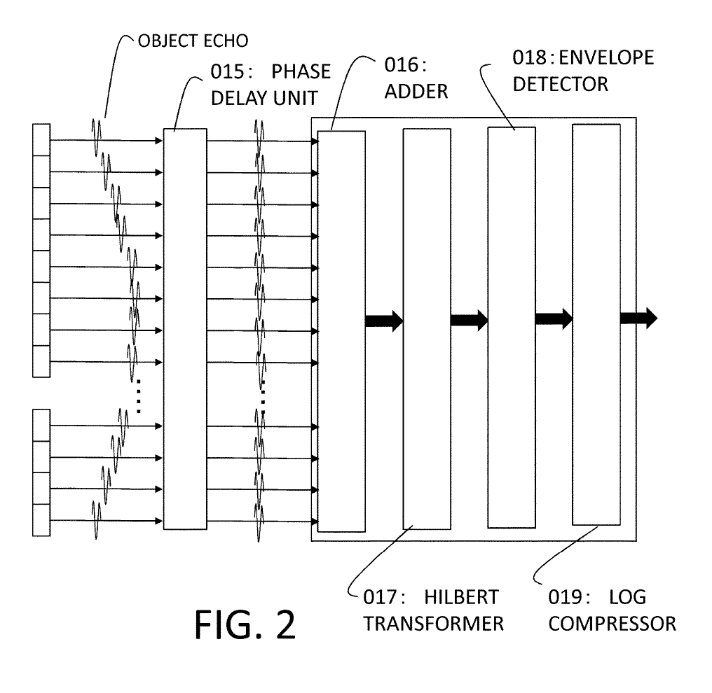

FIG. 2 illustrates a configuration of the signal processor 011. Reference numeral 015 is a phase delay unit that aligns the phases of signals received by the respective conversion elements. Reference numeral 016 is an adder that adds the respective delayed signals. Reference numeral 017 is a Hilbert transformer that applies Hilbert transform to the added signal, and reference numeral 018 is an envelope detector for detecting the envelope. Reference numeral 019 is a LOG compressor that applies LOG compression to the signal obtained by the envelope detection.

(Image Reconstruction)

First, image reconstruction in which ultrasound waves are transmitted to the object 001 and an echo signal from the object 001 is converted into an image will be described with reference to FIG. 1.

The transmission controller 010 determines a delay time when the respective conversion elements 004 that form a transmission opening are driven in order to form a transmission beam at an arbitrary position. The transmission controller 010 transmits an electrical signal as a control signal to the respective conversion elements 004 based on the determined delay time. The respective conversion elements 004 transmit ultrasound waves to the object 001 via the holding member 002 according to the control of the electrical signal.

The holding member 002 is preferably formed of a material having an acoustic impedance difference from the object 001 and the matching material 005 in order to transmit acoustic waves. Moreover, a member capable of pressing the object 001 to maintain the shape of the object 001 such as a member having high rigidity or a member having flexibility is preferred in order to hold the object 001. Examples of a member having high rigidity include a resin material such as PET, polymethylpentene, or acryl. Examples of a member having flexibility include a material such as a rubber sheet such as latex or silicon and urethane.

The thickness of the holding member 002 is preferably as small as possible so that attenuation of acoustic waves passing through the holding member 002 does not increase. Thus, the thickness is preferably between approximately 50 μm and 1 mm to keep balance with the rigidity required for holding the object 001.

The matching material 005 preferably propagates acoustic waves and does not interfere scanning of the probe 003. Examples of the matching material 005 include liquid such as water, DIDS, PEG, silicon oil, or castor oil. The matching material may be stored in a container such as a water tank having an opening through which the holding member is inserted.

The shape of the holding member is not limited to a hemispherical cup shape. Examples of a shape close to a hemisphere include a spherical crown shape, a spherical zone shape, a shape obtained by cutting a portion of an ellipse, other curved surface shape, and a shape obtained by combining curved surfaces and planar surfaces. A cup shape of which the central portion protrudes toward the probe is preferred.

Ultrasound waves transmitted from the transmission opening formed by the respective conversion elements 004 are reflected and scattered by the object 001 and return to the conversion elements 004 as ultrasound echoes. Among the ultrasound echoes, an ultrasound echo received by a plurality of groups of conversion elements 004 that forms a reception opening is converted to an analog electrical signal and is output as a reception signal.

The reception signal is reconstructed as an image by the signal processor 011. The details of this process will be described with reference to FIG. 2.

The phase delay unit 015 determines a delay time of the reception signal based on depth information and performs a process of delaying the respective reception signals. The delayed reception signals are added by the adder 016. The added signal is subjected to Hilbert transform in the Hilbert transformer 017 and envelope detection in the envelope detector 018 whereby an image is reconstructed. In this example, the processing of the signal processor 011 uses a phasing addition method which is generally used in an ultrasound diagnostic apparatus. However, other reconstruction methods such as adaptive signal processing may be used. The reconstructed image data is LOG-compressed by the LOG compressor 019, and one line of image data is obtained. A series of processes are performed while moving the scanning line whereby a two-dimensional ultrasound image along the scanning direction is created.

(Driving of Probe and Imaging Method)

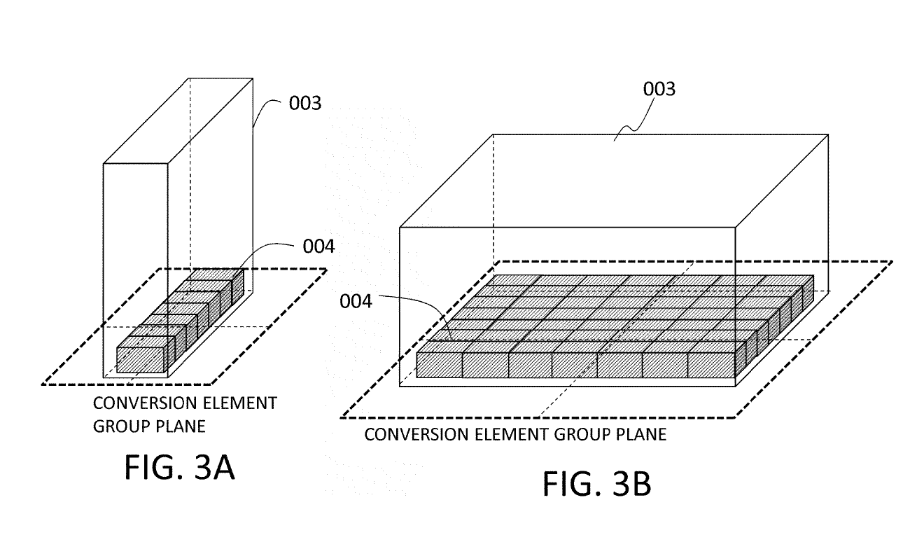

The probe 003 will be described with reference to FIGS. 3A and 3B. An optional conversion element 004 may be used as long as the conversion element can convert ultrasound waves to electrical signals. For example, a conversion element having relatively high conversion efficiency such as PZT, PVDF, or cMUT elements is preferred. A piezoelectric element such as PZT has a structure in which the element is sandwiched between electrodes and expansion and contraction of the piezoelectric element is converted to electrical signals. A matching layer and an acoustic lens are provided on a surface in which ultrasound waves are transmitted and received, and a backing material is provided on the opposite surface to improve the transmission and reception conversion efficiency of ultrasound waves. In the present specification, a surface in which a plurality of conversion elements of a probe are arranged and ultrasound waves are transmitted and received will be defined as an “element plane”. A photoacoustic apparatus described later does not require a ultrasound wave transmitting function. However, in an apparatus which serves as both an ultrasound echo apparatus and a photoacoustic apparatus, the ultrasound transmission and reception probe may have a photoacoustic wave receiving function.

The probe 003 has the plurality of conversion elements 004 which are arranged in series (see FIG. 3A) or planarly (see FIG. 3B) to form an array. A series-type probe will be referred to as a 1D probe, and a planar-type probe will be referred to as a 2D probe. In the case of the 2D probe, the element plane indicates a surface in which the conversion elements are arranged literally. In the case of the 1D probe, a surface of which the normal line corresponds to a direction in which a directional angle of the elements is the highest is referred to as an element plane. A “plane” in relation to a layout of elements does not strictly indicate a two-dimensional plane but may have a width in a three-dimensional direction depending on the shape of the probe, element characteristics, or directivity. The same is true for a plane (“scanning plane”) on which the probe scans as well as the element plane.

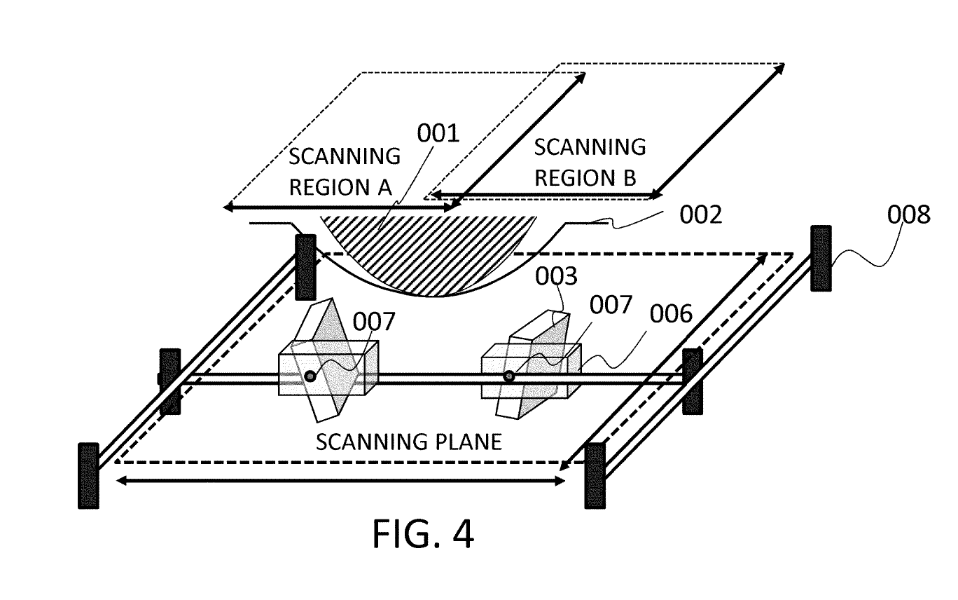

The driving of the probe 003 and the imaging method will be described with reference to FIG. 4. The probe 003 is attached to the carriage 006 and is moved along a two-dimensional scanning plane that faces the holding member 002 by the driving mechanism 008. For example, a combination of a pulse motor and a ball screw, a linear motor, and the like is preferably used as the driving mechanism 008, but the driving mechanism 008 is not limited to this.

The probe 003 is fixed at an optional inclination angle by the inclination adjustment mechanism 007 provided in the carriage 006. A gonio stage and a rotation stage capable of adjusting the inclination angle can be preferably used as the inclination adjustment mechanism 007. When it is necessary to change the inclination angle during imaging depending on the condition of the holding member 002, an automatic stage including a driving system such as a stepper motor is effectively used. Moreover, when the inclination angle of the probe 003 is defined depending on the condition of the holding member 002, the angle may be set by a manual stage or a jig that defines the angle may be used instead of using a stage.

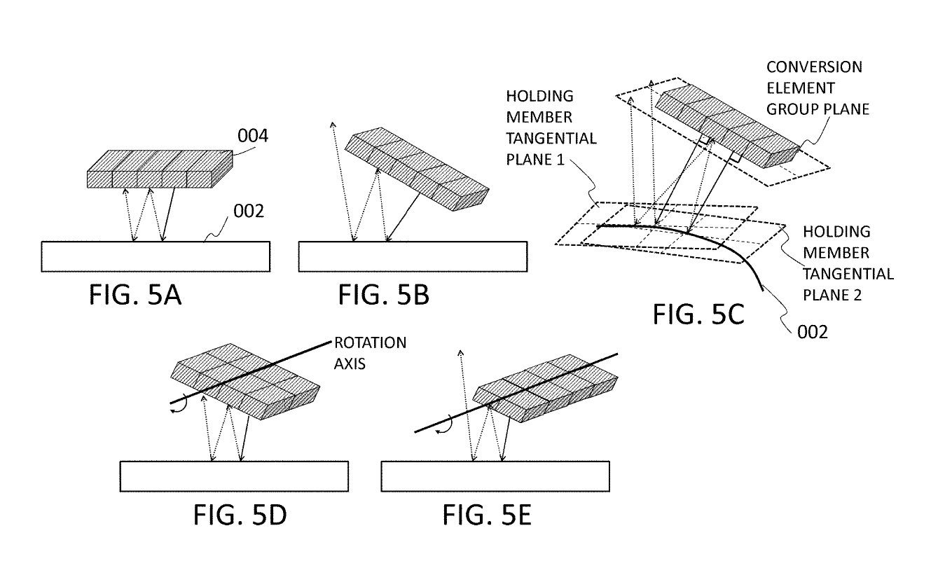

The reason why the probe 003 is inclined is to suppress multiple reflections occurring between the holding member 002 and the conversion element 004 via the matching material 005. This principle will be described with reference to FIGS. 5A to 5E. FIGS. 5A to 5E illustrate a relative positional relation between the group of conversion elements 004 included in the probe 003 and the holding member 002. In FIGS. 5A to 5E, arrows indicate ultrasound waves propagating between the conversion element and the holding member. Although the actual holding member 002 has a curvature, the holding member 002 is approximately illustrated by a plane in FIGS. 5A to 5E for better understanding.

As illustrated in FIG. 5A, when an extension plane of the element plane is parallel to an extension plane of the holding member surface, a generated multiple reflection signal is likely to remain in the space between two planes. In contrast, as illustrated in FIG. 5B, when the extension plane of the element plane crosses the extension plane of the holding member surface, a multiple reflection signal propagates in a direction away from the group of conversion elements 004.

Although simplified in FIGS. 5A and 5B, the actual holding member 002 has a curvature that follows the shape of the object 001. Thus, as illustrated in FIG. 5C, when all tangential planes of the holding member in the normal direction of the element plane cross the extension plane of the element plane at the respective positions to which the element plane has moved with scanning of the probe, it is possible to suppress multiple reflections. In other words, the probe needs to be inclined so that the element plane is not parallel to the tangential plane of the holding member at a position facing the element plane. In FIG. 5C, two tangential planes at the intersection between the normal line from a point on the element plane and the surface of the holding member are illustrated. In the present invention, the inclination of the probe is controlled so that all tangential planes in a region on the holding member facing the element plane cross the extension plane of the element plane.

Here, since the holding member has a curvature, an appropriate inclination angle changes depending on the scanning position of the probe. Thus, in the following embodiments, a plurality of probes is provided or a mechanism for changing the inclination of the probe is provided.

Moreover, the region on the holding member facing the element plane is not necessarily limited to an orthographic projection from the element plane but may have a certain degree of stretch depending on the directivity of elements. The region on the holding member facing the element plane indicates a region on the holding member being in the normal direction from a point within a region on the element plane. All tangential planes of the region on the holding member may be discussed as the tangential plane, and a portion outside the range of the orthographic projection from the element plane may not be discussed.

The multiple reflection suppression effect changes depending on an inclination direction of the probe 003. This will be described with reference to FIGS. 5D and 5E. A large number of probes 003 has long and short sides depending on the size and arrangement of the conversion elements 004. Even if inclination angles are the same, acoustic waves can rarely escape the group of conversion elements 004 illustrated in FIG. 5D in which the probe is inclined about a rotation axis parallel to the short side, whereas acoustic waves can easily escape the group of conversion elements 004 illustrated in FIG. 5E in which the probe is inclined about a rotation axis parallel to the long side. Thus, the probe is preferably inclined about the rotation axis parallel to the long side as illustrated in FIG. 5E.

From the above, a preferable inclination angle is defined by the following two conditions.

(1) An extension plane of the element plane crosses (is not parallel to) all tangential planes of the region on the holding member facing the element plane.
(2) The element plane and the tangential plane are inclined about a rotation axis parallel to the long side of the element plane.

The inclination adjustment mechanism 006 fixes the probe 003 so that an inclination angle that satisfies the two conditions is created. The inclination angle at which multiple reflections are suppressed is determined by the following factors.

(a) The acoustic impedances of the conversion element 004, the holding member 002, and the matching material 005.
(b) The lengths of the short sides of the conversion element 004 and the probe 003.
(c) The distance between the conversion element 004 and the holding member 002.

When the above factors are determined, it is possible to calculate the influence on output images, of artifacts resulting from multiple reflections by simulations or tests. When the inclination angle is increased, the multiple reflection suppression effect increases, whereas the reception sensitivity may decrease due to directivity. Thus, it is sufficient to set the inclination angle such that the influence on images, of artifacts decreases to be smaller than a predetermined threshold. This threshold may preferably be set to a value input by a user. Moreover, depending on the distance between the conversion element 004 and the holding member 002, the echo signal of multiple reflections may arrive outside an image data acquisition period. In this case, since artifacts are not displayed in an image, the inclination angle may be such an angle that the multiple reflection intensity is within a display level.

The holding member 002 preferably has a shape having a curvature that follows the shape of the object 001. Thus, the ideal inclination angle of the probe 003 in all imaging regions is different from position to position. Thus, as illustrated in FIG. 4, the scanning region is divided and the inclination angle of the probe 003 is changed. The number of divided scanning regions, the range of each scanning region, and the inclination angle of the probe in each scanning region are determined by the configuration of the holding member 002. The configuration means the shape of the holding member 002 when holding the object 001 and an acoustic impedance.

When the driving controller 009 controls the driving mechanism 008 based on an imaging range that is designated by a user or determined in advance, the probe moves within the scanning region. Moreover, the inclination angle is adjusted typically by an inclination controller 020 (described later) that drives the inclination adjustment mechanism 007. Moreover, as an inclination angle adjustment method, a method of preparing a plurality of probes 003 and defining the scanning region and the inclination angle of each probe 003 is known. In the case of the latter method, it is necessary to define the scanning region and the inclination angle of each probe 003 based on the known configuration of the holding member 002 and the acoustic characteristics of the matching material 005.

(Creation of Three-Dimensional Image)

The signal processor 011 and the three-dimensional image synthesizer 012 performs a series of image reconstruction processes based on the acoustic waves acquired by the probe 003 in respective scanning regions to acquire a plurality of items of three-dimensional image data. The three-dimensional image synthesizer 012 arranges the items of three-dimensional image data so as to correspond to the coordinate positions of the respective scanning regions. Since the probe 003 is inclined with respect to the scanning plane, the group of items of acquired image data is arranged in a coordinate system distorted in the inclination direction.

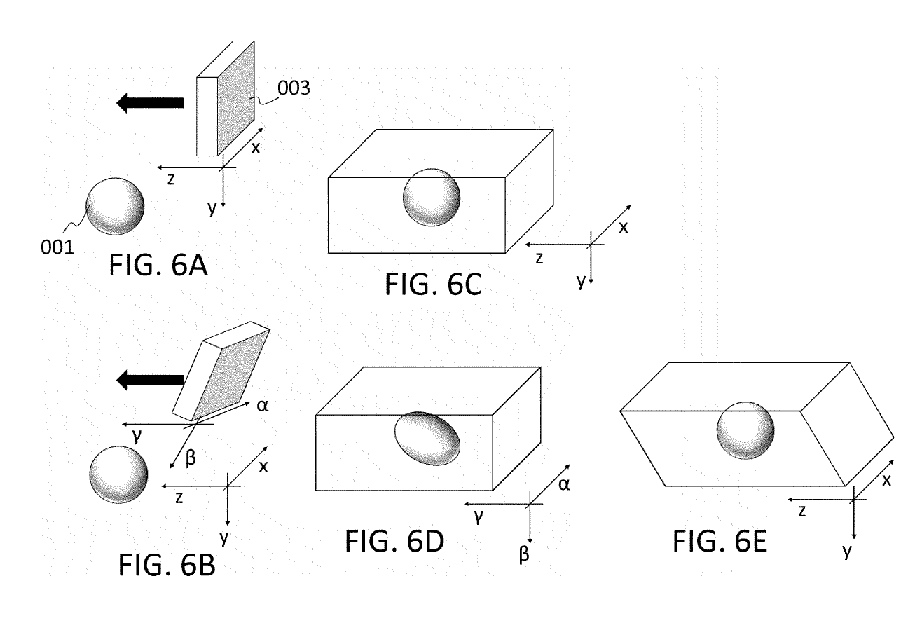

This will be described with reference to FIGS. 6A to 6E. FIGS. 6A and 6B illustrate how image data is acquired while the probes 003 having different inclination angles move on the object 001 in the direction indicated by bold arrows. FIGS. 6C and 6D illustrate items of three-dimensional image data in which the items of image data acquired in FIGS. 6A and 6B are arranged in the moving direction of the probe 003.

In FIG. 6A, for actual space coordinates xyz, x corresponds to a B-mode scanning direction, y corresponds to a beam forming direction, and z corresponds to a probe scanning direction. In this case, as illustrated in FIG. 6C, the acquired data arrangement corresponds to the actual space coordinates xyz.

On the other hand, in FIG. 6B, the probe 003 is inclined, and α corresponds to the B-mode scanning direction, corresponds to the beam forming direction, and γ corresponds to the probe scanning direction. In this case as illustrated in FIG. 6D, the acquired data arrangement corresponds to acquisition data space coordinates αβγ. Since the image data arrangement illustrated in FIG. 6D does not correspond to the actual space coordinates xyz, the image is displayed in a distorted state. In this case, although it is assumed that the beam forming direction is vertical to the element plane, when beam steering is performed, the acquisition data space coordinates αβγ change by the inclination thereof.

In order to display images so as to correspond to the actual space coordinates, as illustrated in FIG. 6E, it is necessary for the image converter 013 to convert the acquired three-dimensional image data to the actual space coordinates. As an image conversion method, a method of repeating two-dimensional affine transform and a method of converting the acquired three-dimensional image data to coordinate values calculated from the inclination angle of the probe 003 or the distance from the rotation axis may be used. Although an approximation method such as a bicubic method is preferred as an image data interpolation method, a nearest neighbor method or a bilinear method may be used.

The image converter 013 creates a plurality of items of three-dimensional image data converted to the actual space coordinates. These items of three-dimensional image data are combined by the image combiner 014, whereby a three-dimensional image of the all scanning region is created. When the range of the scanning region is set, it is preferable to prepare a superimposed region in the respective combined three-dimensional image portions corresponding to the respective scanning regions. By doing so, it is possible to make the boundary portion invisible when the images are combined. As a method of superimposing the combined portions, although it is preferable to apply a weight according to the distance from the center of the superimposed region of the respective scanning regions, the same weight may be applied.

The image display unit 021 displays the image data combined by the image combiner 014. As the image display unit 021, a liquid crystal display, a plasma display, an organic EL display, a FED, and the like can be used. The image display unit 021 may not necessarily be mounted on the system. The present system may create image data only and transmit the image data to another image display device to allow the image display device to display images.

According to the method of controlling the angle of the probe and scanning the probe and the method of setting the scanning region, it is possible to suppress multiple reflections associated with transmission of ultrasound waves and reception of echo waves. Thus, it is possible to display images helpful to diagnosis with few artifacts.

### First Embodiment

### Apparatus Configuration

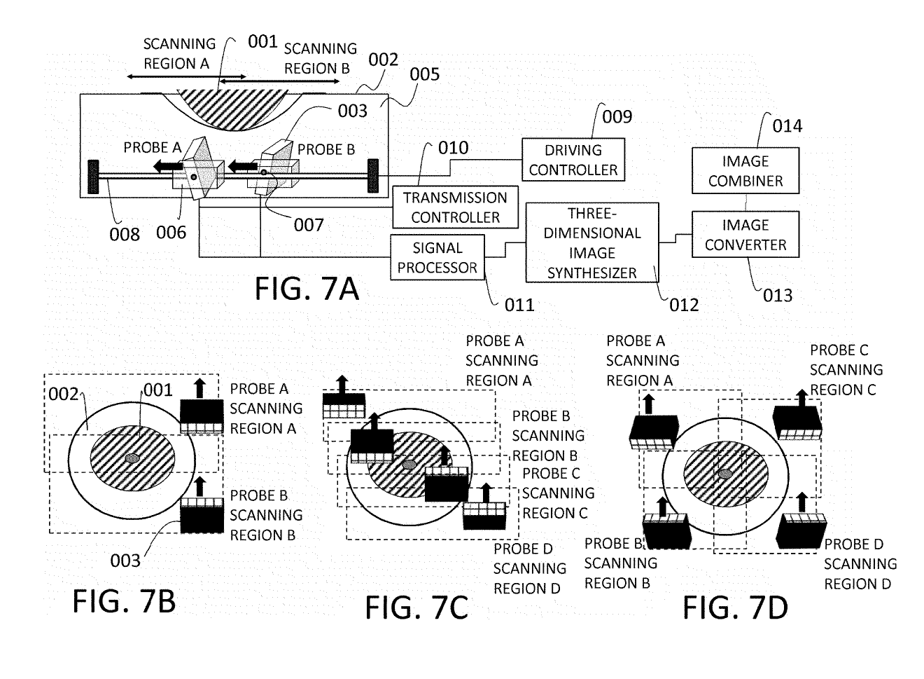

FIGS. 7A to 7D illustrate schematic diagrams of a system of an object information acquiring apparatus according to the present embodiment. A measurement target is an object 001 such as the breast of a living body. The object information acquiring apparatus includes a holding member 002, a ultrasound probe 003, a matching material 005, a carriage 006, an inclination adjustment mechanism 007, and a driving mechanism 008. The apparatus further includes a driving controller 009, a transmission controller 010, a signal processor 011, a three-dimensional image synthesizer 012, an image converter 013, and an image combiner 014.

The constituent elements ranging from the driving controller to the image combiner may be realized by an information processing apparatus which includes a processor, a storage device, a memory, an input/output unit, and the like and operates according to a program and may be realized by a circuit having the functions of the respective constituent elements. In the former case, an actual program module configuration is not particularly limited. Moreover, the program is not limited to be integrated into the apparatus, but the program may operate, for example, in a plurality of apparatuses connected online. Moreover, the lines connecting respective blocks in the drawing are drawn for the sake of convenience to illustrate transmission and reception of electrical and control signals necessary for describing the drawings. The same is true for the respective embodiments described below.

(Operation of Apparatus)

First, an ultrasound imaging apparatus will be described. Two 256-ch and 1D linear probes were used as the ultrasound probe 003. The conversion elements 004 that form the probe 003 were PZT having a central frequency of 8 MHz and a size of 4 mm. The respective elements were arranged in a line so that a lateral pitch was 0.2 mm.

A 0.5 mm-thick hemispherical bowl-shaped member formed of a PET resin and having a size capable of receiving one breast was used as the holding member 002 for holding the object. It is preferable to prepare a plurality of holding members and to replace the holding member according to the size and the shape of the breast. Water was used as the matching material 005 and was circulated by a pump so that the water temperature was maintained to be near 35° C. using a heater. The reason why the water temperature was maintained to be near 35° C. is to prevent an examinee from feeling discomfort and to define the speed of sound in the matching material 005 to improve the image reconstruction accuracy. The water temperature control method is not limited thereto, and other methods may be used. Moreover, although it is preferable to control the water temperature from the perspective of improving the reconstruction accuracy, since such control is not essential, the matching material 005 that is stable at the room temperature may be used.

The transmission controller 010 transmits an electrical signal (control signal) for forming an ultrasound wave transmission focus on an arbitrary position to the respective conversion elements 004. The control signal is transmitted to the object 001 by being converted to ultrasound waves by the respective conversion elements 004. The ultrasound wave is reflected and scattered by the object 001 and is received again by the plurality of conversion elements 004 that form an reception opening as ultrasound echoes. In the present embodiment, the reception opening is formed by a group of 64 conversion elements 004, and the respective reception signals are transmitted to the signal processor 011 and are subjected to phasing addition, Hilbert transform, envelope detection, and LOG compression, whereby the image data on the scanning line is reconstructed. The scanning line is moved in the lateral direction of the probe 003 whereby two-dimensional image data is reconstructed.

Next, a probe driving method will be described.

In the examples of FIGS. 7A to 7D, two ultrasound probes 003 was provided on different carriages 006 at different inclination angles and scanned different scanning regions. FIG. 7B is a view of the holding member 002 when seen from the probe 003. Two scanning regions A and B are set so that a superimposed region is formed in the central portion of the holding member and are scanned by two probes A and B. In the present embodiment, ultrasound waves are transmitted and received during main scanning by defining an elevation direction of the probe 003 as a main scanning direction and the lateral direction of the probe 003 as a sub-scanning direction.

The inclination adjustment mechanism 007 rotates the respective probes 003 about a rotation axis parallel to the sub-scanning direction. The element plane of each probe does not face the central side of the holding member but is inclined toward the outer side. However, since it is not possible to transmit and receive ultrasound waves to and from the object if the orthographic projection of the element plane is completely outside the holding member, the inclination angle needs to be in a range in which the holding member is included in the position facing the element plane. The inclination angle is determined by a directional angle at which the respective conversion elements can receive acoustic waves with significant intensity and an angle at which an artifact suppression effect is obtained, and is preferably determined by simulations and tests.

In the present embodiment, a left-right direction of the drawing sheet of FIG. 7A is used as a rotation axis, and the probes A and B have an inclination angle of 15° and −15°, respectively, about the rotation axis. The probes 003 are provided so that the shortest distance from the holding member 002 is 10 mm. The inclination angle is calculated from the acoustic impedances of the conversion element 004, the matching material 005, and the holding member 002, a width (4 mm) of the conversion element 004, and the distance (the shortest distance 10 mm) between the conversion element 004 and the holding member. The acoustic impedance is 30, 1.5, and 3 [Mega Rayls] for PZT, water, and PET, respectively. In the present embodiment, the inclination angle of 15° is calculated from simulation results and test values, and the optimal inclination angle changes depending on the conditions.

Moreover, in the present embodiment, although the scanning region was divided into two sub-scanning regions A and B using two probes A and B, the number of divided regions and a method of setting the regions are not limited thereto. For example, FIGS. 7C and 7D illustrate an example in which four different sub-scanning regions A, B, C, and D are set using four probes A, B, C, and D.

In FIG. 7C, the entire scanning region are divided into four regions in the main scanning direction. In this case, the inclination angle of the probes A and D may be set to be smaller than that of the probes B and C. This is because the more the region located closer to the outer side, the larger the inclination angle of the holding member tangential plane, and the larger than angle between the surface of the holding member and the scanning plane, the less likely the multiple reflections occur even when the inclination angle of the probe is small. For example, the inclination angles are set to 10°, 15°, −15°, and −10° for the probes A, B, C, and D, respectively. This is because the sub-scanning regions A and D have a large crossing angle between the scanning plane and the tangential plane of the holding member and have a large distance between the group of conversion elements 004 and the holding member 002, and thus the influence of multiple reflections can be decreased even when the inclination angle of the probe 003 is small.

In FIG. 7D, the scanning region in FIG. 7B is further divided into two regions in the sub-scanning direction. Moreover, the probe is inclined in the main scanning direction about the rotation axis parallel to the long side of the group of conversion elements 004 and is also inclined in the sub-scanning direction about the rotation axis parallel to the short side. In the embodiment, the inclination angle in the main scanning direction is set to ±15° similarly to FIG. 7B and the inclination angle in the sub-scanning direction is set to 15° so that the element plane faces the central portion of the holding member 002. The inclination angle in the sub-scanning direction is set to such a value that the transmission and reception beams are incident approximately at an angle vertical to the holding member 002. By setting the angle in such a manner, the influence of refraction, reflection, and the like on the holding member surface based on the Snell's law is reduced. Thus, once a minimum inclination angle necessary for reducing multiple reflections is secured, it may be preferable to allow as many transmission and reception beams as possible to be incident vertically on the holding member 002. By the same reasons, the number of divided scanning regions is increased in FIGS. 7C and 7D. However, when the number of sub-scanning regions increases, the driving control and the image processing become complex, which requires careful attention.

The carriage 006 is moved by the driving mechanism 008. In the present embodiment, a pulse motor is used as a motor. The carriage 006 is moved at an arbitrary velocity to an arbitrary position in a biaxial direction by the driving mechanism 008 obtained by combining a pulse motor and a ball screw. Moreover, the carriage 006 is set so as to scan the sub-scanning regions A and B by the driving controller 009.

The three-dimensional image synthesizer 012 arranges two-dimensional image data so as to correspond to coordinate positions to create three-dimensional image data in the sub-scanning regions A and B. The image converter 012 converts the items of three-dimensional image data to the actual space coordinates in order to absorb the influence of a difference in the inclination angle of the probe 003 between these items of three-dimensional image data. This conversion process will be described with reference to FIGS. 6B, 6D, and 6E. FIG. 6B illustrates how image information of the probe 003 inclined by a predetermined angle is acquired in the actual space coordinates xyz. FIG. 6D illustrates image data which originates from the acoustic waves received by the probe illustrated in FIG. 6B and is arranged by the three-dimensional image synthesizer 012. FIG. 6E illustrates image data converted to the actual space coordinates xyz by the image converter 013 from the image data illustrated in FIG. 6D.

Here, combination of items of image data of sub-scanning regions (typically, sub-scanning regions in which the inclination angles of the probes are different) requires conversion (conversion to the actual space coordinates) to FIG. 6E. In the present embodiment, coordinate conversion was performed using the equation of rotational coordinates corresponding to the inclination angle and the distance about the rotation axis of the probe 003. As a result of the coordinate conversion, interpolation is required to extract the data in the acquisition data space coordinates. In the present embodiment, reference values were calculated using a method of interpolating 64 items of image data around the acquisition data space coordinates α, β, and γ corresponding to the actual space coordinates xyz according to a cubic equation like a bicubic interpolation in the directions of the three axes α, β, and γ.

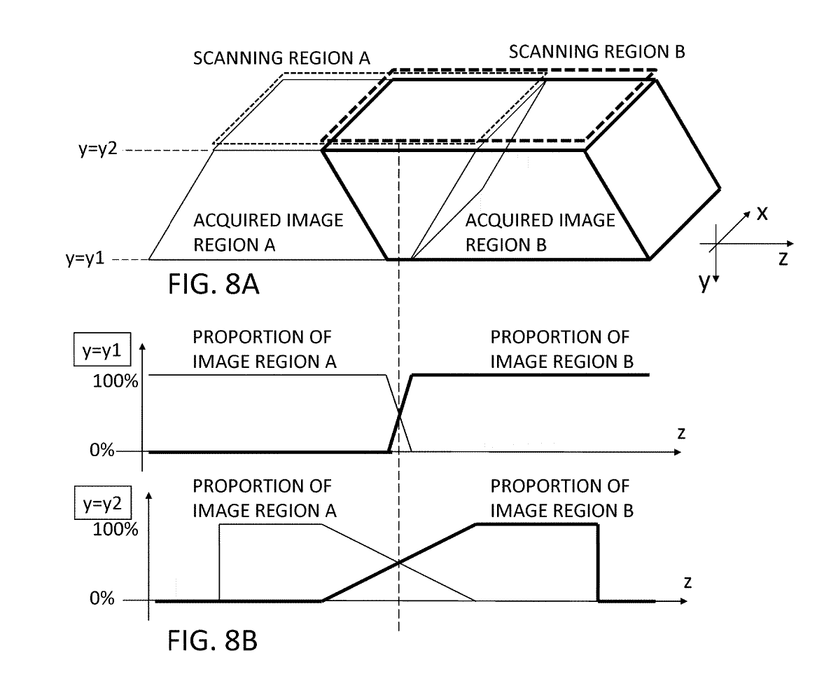

The image combiner 014 combines the three-dimensional image data converted to the actual space coordinates in the superimposed region positioned at the boundary portion between the sub-scanning regions to create three-dimensional image data of the entire scanning region. Image processing in the superimposed region employed in the present embodiment will be described with reference to FIGS. 8A and 8B. FIG. 8A illustrates acquired image regions A and B in the actual space coordinates acquired in the sub-scanning regions A and B. A region in which the acquired image regions A and B are superimposed is the superimposed region. FIG. 8B illustrates a combination ratio of two items of image data at the z-coordinates corresponding to y=y1 and y2. The weighting ratios of the two items of image data at the central coordinate of the superimposed region are set to 50% and the weight changes with the distance from the central coordinate. By using such a weight, it is possible to obtain a combined image in which the boundary is not visible. When the scanning region is divided as illustrated in FIG. 7D, although the images in the x-direction (the sub-scanning direction) need to be combined, the images may be combined in a similar manner.

Moreover, the images of the sub-scanning regions in the x-direction may be combined using the same weighting ratios as the z-direction. This is because these images can be combined in the same acquisition data space coordinates, by collecting image data in the same scanning region in advance, the processes of converting the images to the actual space coordinates in the image converter 013 can be performed collectively.

With the above-described process, the three-dimensional image data of the entire imaging region is obtained. This three-dimensional image data allows an arbitrary sectional image to be seen on a liquid crystal display.

As described above, by appropriately adjusting the angle of the probe, it was possible to suppress artifacts resulting from multiple reflections when the breast held by the cup-shaped holding member 002 was imaged to generate a three-dimensional ultrasound image. Moreover, it was possible to prevent a sense of incongruity in the superimposed portion when the imaging results obtained in the plurality of sub-scanning regions are combined.

### Second Embodiment

In the present embodiment, the configuration and the basic operation of the apparatus are the same as those of the first embodiment. A main difference is that only one probe 003 is used and the inclination controller 020 is present in the present embodiment. The inclination controller 020 controls the inclination adjustment mechanism 007 in order to change an inclination amount of the probe 003 in respective sub-scanning regions. An automatic rotating stage having a stepping motor was used as the inclination adjustment mechanism 007.

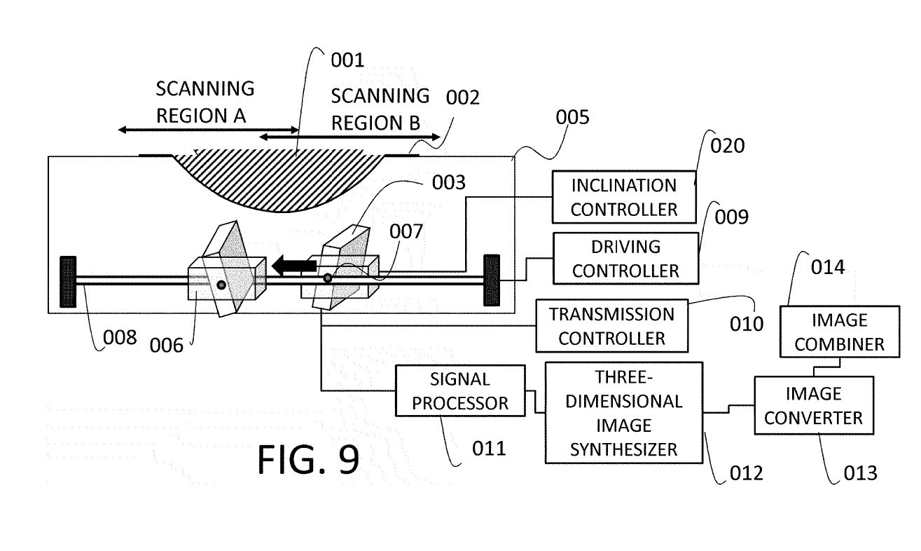

The details of the embodiment will be described with reference to FIG. 9.

First, the inclination controller 020 drives the inclination adjustment mechanism 007 to set the inclination angle of the probe 003 to 15°. In this state, the image data of the sub-scanning region A is acquired and the processes up to the image converter 013 are performed. After that, the inclination controller 020 drives the inclination adjustment mechanism to set the inclination angle of the probe 003 to −15°. In this state, the image data of the sub-scanning region B is acquired and the processes up to the image converter 013 are performed. Two items of three-dimensional image data created in the above processes are combined by the image combiner 014.

The breast held by the hemispherical cup-shaped holding member 002 was imaged using the above-described system. As a result, a three-dimensional ultrasound image of the breast, in which artifacts resulting from multiple reflections were removed was obtained using one probe.

### Third Embodiment

In the present embodiment, the configuration and the basic operation of the apparatus are the same as those of the second embodiment. A main difference is that a holding member characteristic calculating unit 023 and a probe driving calculation unit 024 are present. The holding member characteristic calculating unit 023 calculate the shape and the acoustic characteristics of the holding member 002. The probe driving calculation unit 024 determines a driving method of the probe 003 based on the calculated shape of the holding member 002.

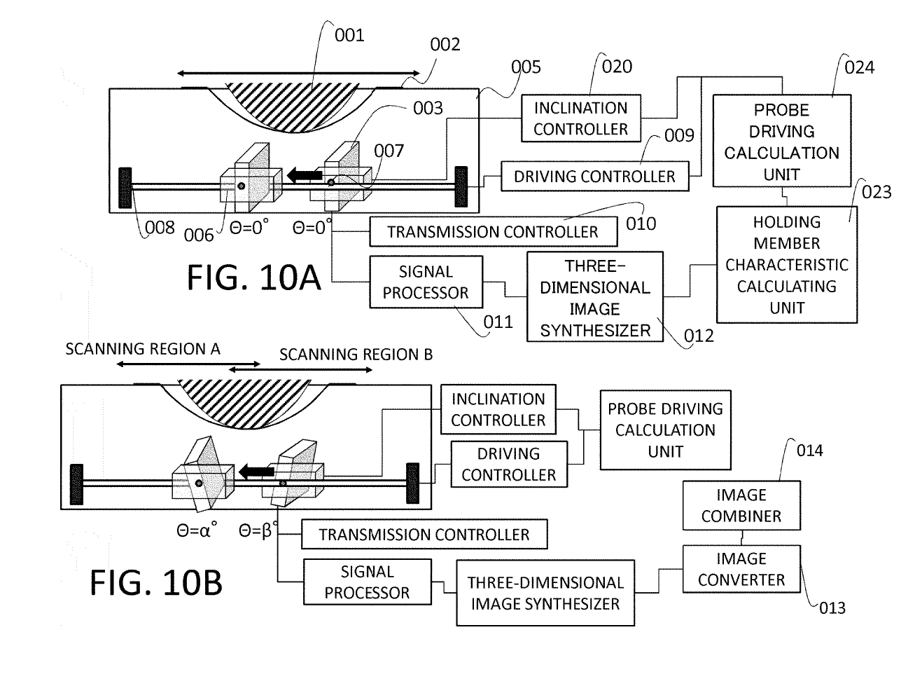

The details of the embodiment will be described with reference to FIGS. 10A and 10B.

In the present embodiment, a silicon rubber sheet of which the shape changes with pressure is used as the holding member 002. Before imaging starts, the breast is pressed against the silicon rubber sheet and a standby is performed until the shape of the sheet becomes stable.

FIG. 10A illustrates a process (a pre-scan process) of acquiring the shape of the holding member before specific information is actually acquired. After imaging starts, first, the inclination angle of the probe 003 is set so that ultrasound waves are transmitted and received vertically to and from the scanning plane and acquires ultrasound signals of the entire imaging region. Moreover, the holding member characteristic calculating unit 023 calculates the shape and the reflection intensity of the holding member 002 based on a reception time and a reception intensity of a strong echo signal acquired for the first time at the respective coordinates.

Subsequently, the probe driving calculation unit 024 determines a scanning region dividing method and the inclination angle of the probe 003 in the respective regions based on the specific information of the holding member, the known shapes and acoustic characteristics of the probe 003 and the conversion element 004, and the known acoustic characteristics of the matching material 005.

The probe driving calculation unit 024 may perform this process based on a correspondence table created by performing simulations and tests in advance using the information on the probe 003 and the characteristics of the matching material 005. In this correspondence table, a multiple reflection avoiding angle is set in correlation with the values of the distance between the conversion element 004 and the holding member 002 and the reflection intensity at the respective coordinates. Moreover, since the shape of the holding member 002 is determined by the holding member characteristic calculating unit 023, it is possible to calculate the angle of the tangential plane of the holding member 002 in the direction normal to the group of conversion elements 004 when the inclination angle of the probe at the respective coordinate was changed. Based on these items of data, the minimum inclination angle of the probe at the respective coordinates within the imaging region is calculated.

The probe driving calculation unit 024 sets a scanning region dividing method and the probe inclination angle in each region based on the minimum inclination angle distribution in the entire imaging region. Preferably, the inclination angle of the probe is set so that the direction of the normal line of the acoustic wave reception surface changes sequentially (the direction of the reception surface changes sequentially).

When a silicon rubber sheet is used as the holding member 002, although the shape thereof changes with pressure, the acoustic characteristics thereof do not change. Thus, the holding member characteristic calculating unit 023 may calculate the shape only and an existing value may be used as the acoustic characteristics of the holding member 002. In this case, the probe driving calculation unit 024 determines the imaging conditions based on the shape only using a correspondence table. By doing so, since the number of conditions in the correspondence table is small, it is possible to simplify the process.

Moreover, when it is possible to detect the shape of the holding member in advance to determine a scanning region dividing method based on the shape information and to calculate the inclination angle of the probe 003, a method other than the above method may be used.

When a PET or the like is used as the holding member, although the PET exhibits a smaller degree of deformation than the silicon rubber sheet, the holding member shape acquisition method according to the present embodiment may be applied. Moreover, since the holding member of PET or the like exhibits a small deformation, a plurality of holding members may be prepared according to the size and the shape of the breast so that the holding member can be replaced in an attachment portion provided in a member that supports an examinee. When the holding member is configured to be replaced in this manner, the numerical values in a correspondence table may be recorded in advance for the respective holding members when creating the correspondence table. Moreover, the object information acquiring apparatus may acquire the type of the holding member based on an IC tag or barcode or a value input by a user and acquire an appropriate correspondence table from a memory.

After the measurement and image generation conditions are determined by the above-described process, actual imaging is performed as illustrated in FIG. 10B. For example, the angles are set such that α=15° and β=−15°. The object information acquiring apparatus performs the process described in the second embodiment according to the conditions determined by the probe driving calculation unit 024 to create a three-dimensional image.

The breast held by the holding member 002 having an unknown form was imaged using the above-described system. As a result, a three-dimensional ultrasound image of the breast in which artifacts resulting from multiple reflections were removed was obtained even when a holding member of which the shape changes like a rubber sheet was used.

### Fourth Embodiment

In the present embodiment, the configuration and the basic operation of the apparatus are the same as those of the third embodiment. A main difference is that an image acquiring unit 025 is present.

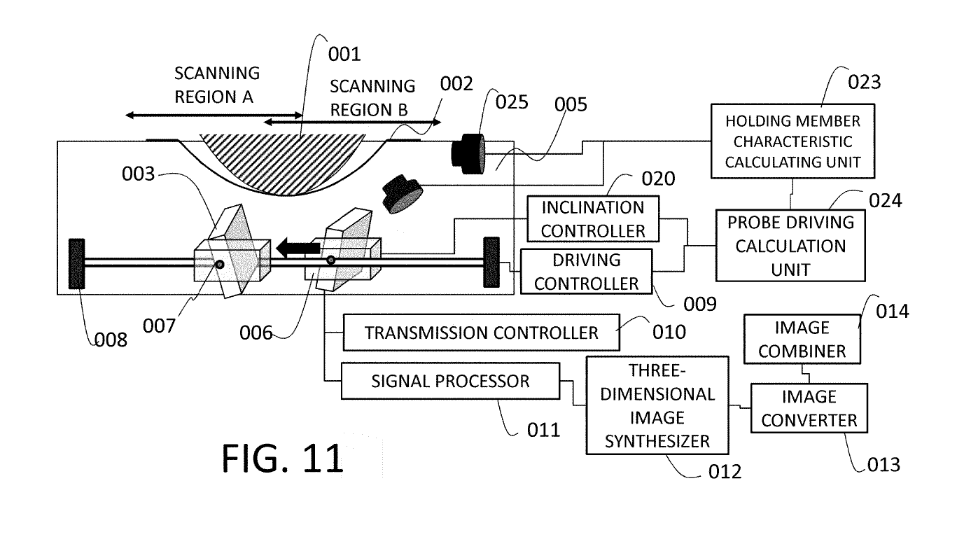

The details of the embodiment will be described with reference to FIG. 11.

Similarly to the third embodiment, a silicon rubber sheet is used as the holding member 002, and standby is performed until the shape of the silicon rubber sheet pressed against the breast becomes stable. In the present embodiment, before imaging starts, the image acquiring unit 025 acquires the image of a side surface of the breast. In the embodiment, two CCD cameras were used to acquire the images of two side surfaces of the breast in the main scanning direction and the sub-scanning direction.

The holding member characteristic calculating unit 023 extracts the shape of the deformed silicon rubber pressed against the breast based on the images acquired by the CCD cameras. Various existing image processing methods (for example, a method of binarizing feature amounts) can be used as the extraction method. Moreover, a three-dimensional shape of the silicon rubber sheet is calculated based on the shape data of the two side surfaces. The type, the position, and the number of image acquiring units are not particularly limited as long as the image acquiring unit can acquire the shape of the holding member. For example, a CMOS camera may be used instead of the CCD camera. Moreover, an entire image of the breast can be effectively acquired when the image acquiring unit is provided on an upper end of a container (support) that forms the probe or near a light emission position. Moreover, the calculation accuracy is improved when the number of image acquiring units is increased or the image acquiring units are provided to cover a wide area.

The probe driving calculation unit 024 determines a scanning region dividing method and the inclination angle of the probe 003 in the respective regions using this information based on the known shapes and acoustic characteristics of the probe 003 and the conversion element 004, and the acoustic characteristics of the matching material 005 and the silicon rubber which is the holding member 002. The subsequent processes are the same as those of the third embodiment.

The breast held by the silicon rubber sheet was imaged using the above-described system. As a result, by calculating the shape of the holding member 002 without performing pre-scanning and setting the probe driving conditions, a three-dimensional ultrasound image of the breast in which artifacts resulting from multiple reflections were removed was obtained.

### Fifth Embodiment

In the present embodiment, the configuration and the basic operation of the apparatus are the same as those of the first to fourth embodiments. A main difference is that the object information acquiring apparatus is not an ultrasound imaging apparatus but is a photoacoustic imaging apparatus.

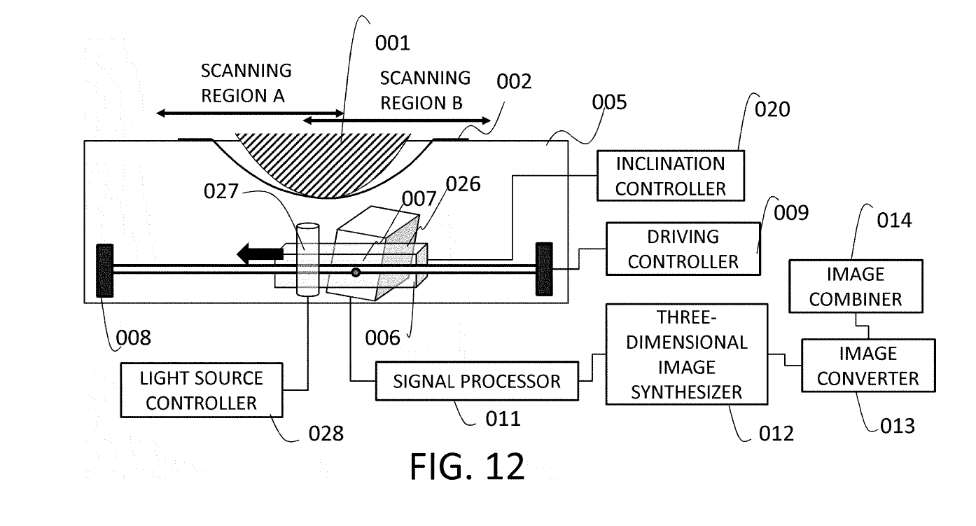

The present embodiment will be described with reference to FIG. 12. A photoacoustic probe 026 and a light source 027 are attached to a carriage 006, and the inclination angle of the probe 026 can be adjusted by an inclination adjustment mechanism 006. The light source 027 is controlled by a light source controller 028.

The photoacoustic imaging apparatus will be described. The light source 027 irradiates an object with a pulse beam. In the present embodiment, a titanium-sapphire laser which is one type of solid lasers is used as the light source. Lasers such as a solid laser, a gas laser, a dye laser, or a semiconductor laser are ideally used as the light source. Moreover, a light-emitting diode may be used instead of the laser. Near-infrared light is preferably used as the light from the light source, and a wavelength between approximately 650 nm and 1100 nm can be used. In the embodiment, a wavelength of 750 nm is used.

The light emitted from the light source 027 propagates through and is absorbed by the object 001. For example, when a living body is irradiated with the above-described near-infrared light, the light is peculiarly absorbed by blood and blood vessels in the living body and acoustic waves are generated due to thermal expansion. When a cancer is present inside a living body, light is peculiarly absorbed by a newborn blood vessel of the cancer and photoacoustic waves are generated.

In the present embodiment, the light source 027 is mounted on the carriage 006 and moved together with the probe 026 so that an imaging portion is effectively irradiated with light. However, the light source 027 may be mounted on a portion other than the carriage 006 as long as necessary photoacoustic waves can be generated. The light source 027 is controlled by the light source controller 028 so as to emit a pulse beam at an interval of 10 Hz.

The photoacoustic probe 026 used in the embodiment includes 600 (20×30) conversion elements having a size of 1 mm by 1 mm. The conversion element 004 uses PZT and the central frequency thereof is adjusted to 2 MHz. The received photoacoustic waves are converted to analog signals by the respective conversion elements 004. The analog signals are reconstructed to image data by the signal processor 011.

A series of processes other than the above processes are the same as those of the first to fourth embodiments.

In the present embodiment, a 2D probe is used. The probe 026 is provided on the carriage 006 so that the short side is parallel to the main scanning direction and the long side is parallel to the sub-scanning direction. The inclination of the probe 026 is adjusted by the inclination adjustment mechanism 007 so that the probe 026 is inclined in the main scanning direction about the rotation axis parallel to the long side. However, the same effect can be obtained by adjusting the inclination adjustment mechanism 007 so that the probe 026 is rotated by 90° to be inclined in the sub-scanning direction. However, in this case, an optimum scanning region dividing method and the inclination angle change depending on the shape or the like of the holding member 002. In any case, the inclination direction of the probe 026 is not limited as long as at least the element plane and the tangential plane of the holding member cross each other so as to be inclined with respect to the rotation axis parallel to the long side of the conversion element group.

An inclination angle calculation method is partially different from a photoacoustic imaging apparatus to an ultrasound imaging apparatus because the artifact propagation paths are different. In the case of an ultrasound imaging apparatus, an ultrasound wave having reciprocated twice or more between the conversion element 004 and the holding member 002 among the ultrasound waves having been transmitted from the conversion element 004 causes artifacts and an inclination angle for reducing the echo signal is set.

On the other hand, in the case of a photoacoustic imaging apparatus, when light is emitted from the light source 027, a vibration occurs in the surface of the holding member 002 and the surface of the probe 026 and an acoustic wave is generated from the surfaces. The acoustic wave having been reflected multiple times between the conversion element 004 and the holding member 002 causes artifacts. Although the acoustic wave generated from the surface of the probe 026 causes artifacts when the acoustic wave reciprocates twice or more like the ultrasound imaging apparatus, the acoustic wave generated from the holding member 002 causes artifacts when the acoustic wave reciprocates 1.5 times or more. Thus, photoacoustic waves tend to increase at an inclination angle at which multiple reflection signals are not received. Moreover, when the length of the short side of the probe 026 is relatively large as in the present embodiment, the inclination angle increases. Thus, it is difficult to realize an inclination angle at which multiple reflection signals are not received completely.

In such a case, it is preferable to set an inclination angle such that an artifact signal is equal to or smaller than a certain threshold. In the present embodiment, the inclination angle is set such that an artifact signal is suppressed to approximately 40 dB for an acoustic signal of the image information of a target object.

Regardless of whether the form of the holding member 002 is known or unknown, similarly to the case of an ultrasound imaging apparatus, a correspondence table is created based on the results of simulations or tests and the inclination angle is determined based on the correspondence table. When the form of the holding member 002 is known, a scanning region dividing method, and the inclination angle of each probe 026 are set in advance. Moreover, when the form of the holding member 002 is unknown, the shape of the holding member 002 is detected by performing pre-scanning or acquiring and analyzing the image of the holding member 002, a scanning range dividing method and the inclination angle of each probe 026 may be determined based on a correspondence table, and images may be obtained.

In the present embodiment, a 1 mm-thick hemispherical cup-shaped holding member 002 formed of polymethylpentene was used. Moreover, as illustrated in FIG. 7B, two sub-scanning regions were provided. The inclination angles of the two probes were adjusted by the inclination adjustment mechanism 007 so that the inclination angles were 10° and −10° for the probes A and B, respectively. In this case, the shortest distance between the probe 0026 and the holding member 002 was set to 10 mm.

The breast held by the holding member 002 was imaged using the above-described system. A three-dimensional ultrasound image of the breast in which artifacts resulting from multiple reflections were reduced was obtained.

As described in the respective embodiments, according to the present invention, it is possible to provide an object information acquiring apparatus that allows a probe to scan an object held by a holding member and is capable of suppressing and reducing multiple reflections occurring between a conversion element plane of the probe and the holding member.

In the present invention, in an object information acquiring apparatus that allows a probe to scan an object held by a holding member having a curvature, the probe is inclined so that an element plane of the probe crosses all tangential planes of the object holding member in the normal direction of a conversion element group of the probe. Due to this, multiple reflections occurring between the element plane of the probe and the object holding member are suppressed.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-234754, filed on Nov. 19, 2014, which is hereby incorporated by reference herein in its entirety.

What is claimed is:

1. An object information acquiring apparatus comprising:
a holding member configured to have a curvature and hold an object;
a conversion element configured to receive an acoustic wave propagating from the object via the holding member and output an electrical signal;
a driving unit configured to move at least one of the conversion element and the holding member so that an acoustic wave reception surface of the conversion element is not parallel to a tangential plane of the holding member at a position at which a normal line of the acoustic wave reception surface drawn from the conversion element crosses the holding member; and
an information processor configured to acquire specific information inside the object using the electrical signal.

2. The object information acquiring apparatus according to claim 1, wherein
the driving unit moves at least one of the conversion element and the holding member so that the acoustic wave reception surface is inclined toward an outer side of the holding member.

3. The object information acquiring apparatus according to claim 1, further comprising:
a probe in which a plurality of the conversion elements are arranged in directions of long and short sides thereof so that the acoustic wave reception surfaces are flush with each other.

4. The object information acquiring apparatus according to claim 3, wherein
the driving unit moves the probe so that the acoustic wave reception surfaces are inclined about a rotation axis parallel to the long side.

5. The object information acquiring apparatus according to claim 1, wherein
the information processor performs a conversion process on the specific information obtained by reconstructing the electrical signal according to an angle between the acoustic wave reception surface of the conversion element and a scanning plane when at least one of the conversion element and the holding member is driven by the driving unit.

6. The object information acquiring apparatus according to claim 1, wherein
the driving unit moves the conversion element in relation to the holding member so that the normal line of the acoustic wave reception surface changes sequentially.
